(19) United States
(12) Patent Application Publication
(10) Pub. No.: US 2026/0102106 A1
(43) Pub. Date: Apr. 16, 2026

(54) DEVICE FOR EXAMINING EYE HEALTH

(71) Applicant: Electronics and Telecommunications Research Institute, Daejeon (KR)

(72) Inventors: Hyung Ju Park, Daejeon (KR); Myungjoon Kwack, Daejeon (KR); Hanvit Kim, Daejeon (KR); Jeong Won Park, Daejeon (KR); Hyeon-Bong Pyo, Daejeon (KR); CHUL HUH, Daejeon (KR)

(21) Appl. No.: 19/275,836

(22) Filed: Jul. 21, 2025

(30) Foreign Application Priority Data
Oct. 15, 2024 (KR) 10-2024-0140666

(51) Int. Cl. A61B 5/398; A61B 3/00; A61B 5/00; A61B 5/266; A61B 5/297

(52) U.S. Cl. CPC A61B 5/398; A61B 3/005; A61B 5/266; A61B 5/297; A61B 5/6803

(57) ABSTRACT

The present invention relates to a device for examining eye health. A device for examining eye health according to the present invention includes a display that blocks external light, generates light stimulation for measurement, and applies the generated light stimulation to an eye of a user, an electrode that comes into contact with a periphery of the eye of the user and measures a biometric signal generated by the light stimulation, and a controller that controls the light stimulation and collect the biometric signal.

### CROSS-REFERENCE TO RELATED APPLICATION

This application claims priority under 35 U.S. C. § 119 to Korean Patent Application No. 10-2024-0140666, filed on Oct. 15, 2024, in the Korean Intellectual Property Office, the disclosure of which is incorporated herein by reference in its entirety.

### BACKGROUND

### 1. Field of the Invention

The present invention relates to a portable biometric signal measuring device including electrodes for measuring a micro biometric signal.

### 2. Discussion of Related Art

An electroretinogram (ERG) device is a device for recording a change in activity potential difference of a retinal nerve cell against light stimulation. This device is used to evaluate a function of a retina to diagnose hereditary retinal diseases and determine causes thereof. Since the change in the activity potential difference of the retinal nerve cells against light stimulation is very small, there are difficulties in that electrodes should be disposed close to eyes to measure the change and a separate light stimulation device should be used in a dark room. Thus, since most conventional electroretinogram examining devices are expensive devices are large in size, and do not provide a user-friendly environment, it is difficult for the general public to conveniently use these devices, and only experienced experts in medical institutions use these devices.

Patent Document 1(US 2022/0004026 A1, Jan. 6, 2022) discloses a system for measuring a retina biometric signal using a contact lens platform, but there is a limitation in that a separate light stimulation device is required in the dark room to measure the electroretinogram.

Patent Document 2(KR 10-2021-0027220 A, Mar. 10, 2021) discloses a contact lens in which a light source and electrodes are integrated to examine an electroretinogram of an eyeball, but there are problems in that a separate means for eyes of a subject that blocks surroundings is required, a process of wearing the contact lens is inconvenient, and the user feels a foreign object.

### SUMMARY OF THE INVENTION

The present invention is directed to providing a device for examining an eye in the form of goggles that a user may carry.

In detail, the present invention is also directed to providing a goggle-type device for examining an eye, which is equipped with a head mounted display capable of controlling external light stimulation for the eye and electrodes capable of measuring a fine biometric signal of the eye and made of a biocompatible conductive hydrogel material including a metal nanomaterial. In the present invention, since the head mounted display is used, separate equipment for controlling light stimulation is not required.

The purpose of the present invention is not limited to the purpose described above, and other purposes which are not described may be understood by those skilled in the art based on the following description.

According to an aspect of the present invention, there is provided a device for examining eye health, the device including a display that blocks external light, generates light stimulation for measurement, and applies the generated light stimulation to an eye of a user, an electrode that comes into contact with a periphery of the eye of the user and measures a biometric signal generated by the light stimulation, and a controller that controls the light stimulation and collects the biometric signal.

The display may include a left-eye display that blocks external light directed to a left eye of the user and applies light stimulation to the left eye and a right-eye display that blocks external light directed to a right eye of the user and applies light stimulation to the right eye.

The electrode may include an active electrode and a ground electrode.

The biometric signal may include an electroretinogram.

The electrode may include an active electrode and a ground electrode, and the active electrode may come into contact with a lower portion of an eyehole of the user.

The ground electrode may come into contact with a forehead of the user.

The biometric signal may include an electroretinogram and a visual evoked potential.

The controller may evaluate the electroretinogram using the visual evoked potential.

The electrode may include a conductive hydrogel material having biocompatibility.

The conductive hydrogel material may include gold nanowires.

The electrode may include a first active electrode, a second active electrode, and a ground electrode, the first active electrode may come into contact with a lower portion of an eyehole of the user and measure the electroretinogram, and the second active electrode may come into contact with a skin surface of any one of a forehead and an the back of the head and measure the visual evoked potential.

The device for examining eye health may have a form of a goggle that comes into contact with a face of the user.

The controller may amplify the biometric signal, convert the amplified signal into a digital signal, and transmit the digital signal to an external terminal through a communication device.

A system for examining eye health may be a system that measures and displays information on eye health of a user. The system may include a device for examining eye health, which measures a biometric signal related to the eye health of the user and a terminal that displays the biometric signal.

The device for examining eye health may include a display that blocks external light, generates light stimulation for measurement, and applies the generated light stimulation to an eye of the user, an electrode that comes into contact with a periphery of the eye of the user and measures the biometric signal generated by the light stimulation, and a controller that controls the light stimulation and collects the biometric signal.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a conceptual view of a system for examining eye health according to an embodiment of the present invention;

FIG. 2 is a block diagram illustrating a configuration of a device for examining eye health according to the embodiment of the present invention; and

FIG. 3 is an exemplary view of the device for examining eye health according to the present invention.

### DETAILED DESCRIPTION OF EXEMPLARY EMBODIMENTS

Advantages and features of the present invention and a method of achieving the advantages and the features will become apparent with reference to embodiments described below in detail together with the accompanying drawings. However, the present invention is not limited to embodiments described below but will be implemented in various forms, and the present embodiments merely make the disclosure of the present invention complete, are provided to completely inform the scope of the present invention to those skilled in the art to which the present invention belongs, and are merely defined by the scope of the appended claims. Meanwhile, terms used in the specification are intended to describe the embodiments and are not intended to limit the present invention. In the specification, a singular form also includes a plural form unless specifically mentioned in a phrase. The term “comprise” or “comprising” used herein means that the presence or addition of one or more other components, steps, operations, and/or elements are not excluded to components, steps, operations, and/or elements described above.

Although the terms “first,” “second,” etc., may be used to describe various components, the components should not be limited by the terms. The terms may be used to distinguish one component from another component. For example, without departing from the scope of the present invention, a first component may be referred to as a second component, and similarly, a second component may be referred to as a first component.

It should be understood that, when it is referenced that a first component is “connected” or “coupled” to a second component, the first component may be directly connected or coupled to the second component, or a third component may be present between the first component and the second component. On the other hand, it should be understood that, when a first component is “directly connected” or “directly coupled” to a second component, a third component is not present therebetween. Other expressions that describe a relationship between components, such as “between” and “directly between” or “adjacent to” and “directly adjacent to,”should be interpreted similarly.

In describing the present invention, when it is determined that detailed description of related widely known technology may unnecessarily obscure the subject matter of the present invention, the detailed description thereof will be omitted.

Hereinafter, embodiments of the present invention will be described in detail with reference to the accompanying drawings. In order to facilitate overall understanding in describing the present invention, the same reference numerals will be used for the same means regardless of the reference numerals.

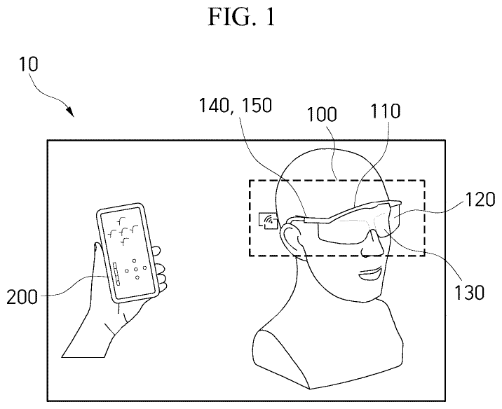

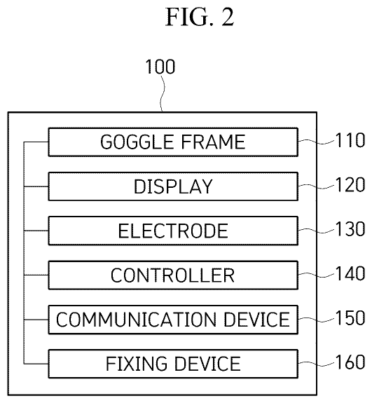

FIG. 1 is a conceptual view of a system for examining eye health according to an embodiment of the present invention, and FIG. 2 is a block diagram illustrating a configuration of a device for examining eye health according to the embodiment of the present invention.

A system 10 for examining eye health according to the embodiment of the present invention includes a device 100 for examining eye health and a terminal 200. The device 100 for examining eye health may include a goggle frame 110, a display 120, an electrode 130, a controller 140, and a communication device 150 and may further include a fixing device 160. The goggle frame 110 allows the device 100 for examining eye health to be in close contact with a face of a user. For example, the goggle frame 110 may include rims that are edges surrounding the displays 120 arranged in individual eyes, a bridge connecting the two rims, temples that are legs of a goggle, hinges connecting the temples and the rims, and temple tips that are ends of the temples and allow the goggle to be fixed to the ears of the user. However, a configuration of the goggle frame 110 is not limited to the above-described example. For example, the temple tip may be changed to a strap. The display 120, the electrode 130, the controller 140, the communication device 150, and the fixing device 160 are mounted on or attached to the goggle frame 110.

The device 100 for examining eye health included in the system 10 for examining eye health may be a device capable of measuring a biometric signal related to eye health and have the form of a goggle to be carried by a user. The device 100 for examining eye health may transmit the measured biometric signal (eye health information) to the terminal 200 in a wired communication manner or a wireless communication manner through the communication device 150. The terminal 200 may display the biometric signal (the eye health information) so that the user recognizes the biometric signal.

Referring to FIG. 2, the device 100 for examining eye health includes the goggle frame 110, the display 120, the electrode 130, the controller 140, the communication device 150, and the fixing device 160. The device 100 for examining eye health illustrated in FIG. 2 is an embodiment, and components of the device 100 for examining eye health according to the present invention are not limited to the embodiment illustrated in FIG. 2, and some components may be added, changed, or omitted as needed.

The displays 120 are mounted on the rims (edges of lens portions for individual eyes in glasses; in the present invention, edge portions of the display 120) of the goggle frame 110. The displays 120 are mounted on the rims of the goggle frame 110 to form a closed space capable of controlling external light stimulation for the individual eyes. That is, the display 120 forms a separate closed space in each of the eyes of the user to form a darkroom environment. Further, the display 120 may stimulate retinal nerve cells of the individual eyes with light.

As another example, the display 120 may be used to measure an eyesight or movement of pupils of the user.

The electrode 130 measures a biometric signal related to the eye health of the user. The electrode 130 is a conductive hydrogel electrode having biocompatibility. The biometric signal measured by the electrode 130 may be an electroretinogram (ERG) or a visual evoked potential (VEP). For reference, the electroretinogram (ERG) refers to a potential caused in the retina by light stimulation, and the visual evoked potential (VEP) refers to an electrical physiological potential caused by visual stimulation and may be measured in the retina or cerebral cortex.

Since a change in the biometric signal, such as the electroretinogram and the visual evoked potential, related to the eye health, is small, in the present invention, a material obtained by adding metal nanoparticles to a hydrogel having biocompatibility may be used as a material of the electrode 130. Conductivity can be increased through the metal nanoparticles. The metal nanoparticles may be gold nanowires. In the present invention, a material having high biocompatibility is used as the material of the electrode 130, a high-performance biometric signal is simply acquired, noise caused by movement is minimized, skin adhesion is increased, and thus precision of examination of the eye health is improved.

The electrode 130 includes an active electrode 131 and a ground electrode 132. Although a position at which the electrode 130 is mounted on the goggle frame 110 is not limited, it is appropriate that the active electrode 131 is disposed in a skin contact portion 111 in contact with a lower portion of an eyehole of the user in the goggle frame 110, and it is appropriate that the ground electrode 132 is disposed in the skin contact portion 111 in contact with a forehead of the user in the goggle frame 110. The skin contact portion 111 may be a rubber pad.

A plurality of active electrodes 131 may be provided, and in this case, may be distinguished by reference numerals. For example, active electrodes in contact with the lower portion of the eyehole may be marked as 131a and 131b, an active electrode in contact with the forehead may be marked as 131c, and an active electrode in contact with the the back of the head may be marked as 131d.

The controller 140 includes a signal amplifier, an analog/digital (A/D) converter, a memory, and a processor. The controller 140 controls light stimulation of the display 120, collects the biometric signal (e.g., the electroretinogram and the visual evoked potential) measured by the electrode 130, amplifies the biometric signal using the signal amplifier, converts the biometric signal into a digital signal using the A/D converter, and transmits the biometric signal converted into the digital signal to the external terminal 200 through the communication device 150. A position at which the controller 140 is disposed in the goggle frame 110 is not limited. For example, the controller 140 may be mounted on the temple portion (eyeglass legs) of the goggle frame 110.

The memory included in the controller 140 may store commands that are executed by the processor, setting values of the light stimulation of the display 120, and biometric signal data measured through the electrode 130.

The processor included in the controller 140 executes the command stored in the memory to control the light stimulation generated in the display 120, stores a digital signal converted from a biometric signal in the memory, and transmits the digital signal to the external terminal 200 through the communication device 150.

The communication device 150 transmits the biometric signal data (eye health information) to the terminal 200 in a wired communication manner or a wireless communication manner under the control of the controller 140. A position at which the communication device 150 is disposed in the goggle frame 110 is not limited, and for example, the communication device 150 may be mounted on the temple portion of the goggle frame 110.

The fixing device 160 may have the form of a temple tip. In this case, the fixing device 160 serves to fix the device 100 for examining eye health to the ears of the user.

As another example, the fixing device 160 may be a strap. For example, the fixing device 160 may be a strap made of a rubber band.

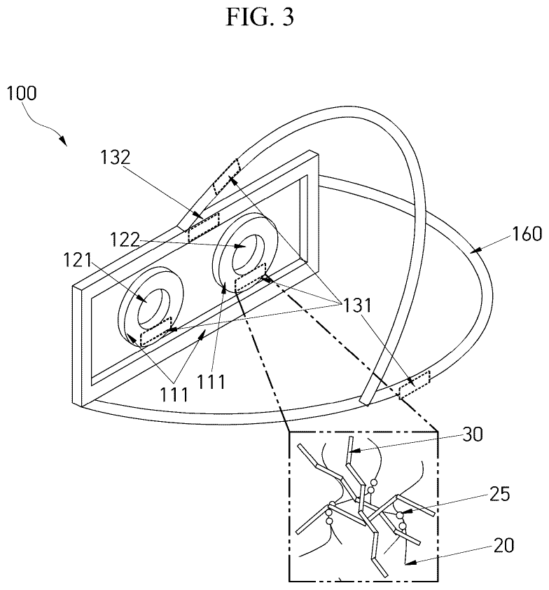

FIG. 3 is an exemplary view of the device for examining eye health according to the present invention.

The device 100 of FIG. 3 for examining eye health includes the goggle frame 110, the display 120, the electrode 130, the controller 140, the communication device 150, and the fixing device 160.

The display 120 includes a left-eye display 121 and a right-eye display 122. The left-eye display 121 blocks external light stimulation directed to the left eye of the user and applies the light stimulation generated for measurement to the left eye. The right-eye display 122 blocks external light stimulation directed to the right eye of the user and applies the light stimulation generated for measurement to the right eye.

The active electrode 131 is disposed in the skin contact portion 111 in contact with the lower portion of the eyehole of the goggle frame 110. The active electrodes 131a and 131b in contact with the lower portion of the eyehole measure the electroretinogram. The ground electrode 132 is disposed on the skin contact portion 111 in contact with the forehead of the user in the goggle frames 110.

The device 100 for examining eye health illustrated in FIG. 3 may measure both the electroretinogram and the visual evoked potential.

To this end, the fixing device 160 includes a rubber band strap for fixing the goggle frame 110 to the face of the user. The rubber band strap is in contact with portions of the forehead and the the back of the head (skin surfaces of the forehead and the the back of the head) of the user, and the active electrodes 131c and 131d for measuring the visual evoked potential are arranged at portions of the rubber band strap, which are in contact with the forehead and the the back of the head (the skin surfaces of the forehead and the the back of the head) of the user.

The controller 140 may collect the electroretinogram and the visual evoked potential measured by the electrode 130 and determine whether the electroretinogram is a normal signal by using the visual evoked potential. The electroretinogram should be recognized by the brain through the optic nerve, and the controller 140 may determine whether the electroretinogram is properly transmitted to the brain using the visual evoked potential.

The controller 140 may communicate with the external terminal 200 such as a personal computer (PC), a laptop computer, or a smartphone through a wired communication manner or a wireless communication manner using the communication device 150 mounted on the goggle frame 110. Further, the controller 140 may allow the device 100 for examining eye health to be used as a device capable of performing various methods of examining eye health to be used as a device capable of performing various eye health examination methods through control of the display 120.

Meanwhile, a conductive hydrogel material having high biocompatibility is used as the material of the electrode 130 of the device 100 for examining an eye health. Examples of hydrogels include polymers such as polyacrylate, polyvinyl alcohol, and gelatin.

As illustrated in FIG. 3, a material having increased conductivity by adding gold nanowires 30 to a structure 25 in which polymers 20 such as polyvinyl alcohol are bonded to each other may be used as the electrode 130 used in the present invention. As a material obtained by adding the gold nanowires to the conductive hydrogel is applied to the electrode 130, even when the electroretinogram is measured on a portion in contact with the lower portion of the eyehole, precision can be increased.

The gold nanowire 30 refers to a wire structure made of gold and having a length in nanometers. In the present invention, a nanowire refers to a structure in which a ratio of a diameter to a length is 1:20 or more, and a nanorod refers to a structure in which a ratio of a diameter to a length is less than 1:20. Since it is not easy to generate the gold nanowires 30 at once by connecting gold particles, it is preferable that gold nanorods are primarily generated by connecting the gold particles, and the gold nanowires 30 are generated by connecting the gold nanorods.

According to the present invention, electrodes made of a biocompatible conductive hydrogel material are used to measure information on eye health, and thus noise caused by movement is minimized, and measurement precision can be improved.

Further, according to the present invention, a display included in a goggle is used as a means for blocking external light stimulation and also used as a light stimulation source, and thus a user can easily acquire information on eye health while carrying a measurement device.

Further, according to the present invention, external light stimulation is blocked using a goggle-type device for examining eye health, and thus a separate dark room is not required for measuring information on eye health.

The effects obtained in the present invention are not limited to the effects described above, and other effects not described will be clearly understood by those skilled in the art to which the present invention pertains from the above detailed description.

Although exemplary embodiments of the present invention have been described above, those skilled in the art may understand that the present invention may be variously modified and changed without departing from the spirit and scope of the present invention described in the appended claims.

What is claimed is:

1. A device for examining eye health, comprising:
a display configured to block external light, generate light stimulation for measurement, and apply the generated light stimulation to an eye of a user;
an electrode that comes into contact with a periphery of the eye of the user and measures a biometric signal generated by the light stimulation; and
a controller configured to control the light stimulation and collect the biometric signal.

2. The device of claim 1, wherein the display includes:
a left-eye display configured to block external light directed to a left eye of the user and apply light stimulation to the left eye; and
a right-eye display configured to block external light directed to a right eye of the user and apply light stimulation to the right eye.

3. The device of claim 1, wherein the electrode includes an active electrode and a ground electrode.

4. The device of claim 1, wherein the biometric signal includes an electroretinogram.

5. The device of claim 1, wherein the electrode includes an active electrode and a ground electrode, and
the active electrode comes into contact with a lower portion of an eyehole of the user.

6. The device of claim 5, wherein the ground electrode comes into contact with a forehead of the user.

7. The device of claim 1, wherein the biometric signal includes an electroretinogram and a visual evoked potential.

8. The device of claim 7, wherein the controller evaluates the electroretinogram using the visual evoked potential.

9. The device of claim 1, wherein the electrode includes a conductive hydrogel material having biocompatibility.

10. The device of claim 9, wherein the conductive hydrogel material includes gold nanowires.

11. The device of claim 7, wherein the electrode includes a first active electrode, a second active electrode, and a ground electrode,
the first active electrode comes into contact with a lower portion of an eyehole of the user and measures the electroretinogram, and
the second active electrode is in contact with a skin surface of any one of a forehead and an the back of the head and measures the visual evoked potential.

12. The device of claim 1, wherein the device for examining eye health has a form of a goggle that comes into contact with a face of the user.

13. The device of claim 1, wherein the controller amplifies the biometric signal, converts the amplified signal into a digital signal, and transmits the digital signal to an external terminal through a communication device.

14. A system for examining eye health, which measures and displays information on eye health of a user, the system comprising:
a device for examining eye health, which measures a biometric signal related to eye health of the user; and
a terminal configured to display the biometric signal,
wherein the device for examining eye health includes:
a display configured to block external light, generate light stimulation for measurement, and apply the generated light stimulation to an eye of the user;
an electrode that comes into contact with a periphery of the eye of the user and measures the biometric signal generated by the light stimulation; and
a controller configured to control the light stimulation and collect the biometric signal.
